(19) United States
(12) Patent Application Publication
(10) Pub. No.: US 2011/0251491 A1
(43) Pub. Date: Oct. 13, 2011

(54) ULTRASOUND TRANSDUCER AND ULTRASOUND DIAGNOSTIC APPARATUS

(75) Inventor: Yutaka OONUKI, Tochigi-ken (JP)

(73) Assignees: TOSHIBA MEDICAL SYSTEMS CORPORATION, Otawara-Shi (JP); KABUSHIKI KAISHA TOSHIBA, Minato-ku (JP)

(21) Appl. No.: 13/084,750

(22) Filed: Apr. 12, 2011

(30) Foreign Application Priority Data
Apr. 12, 2010 (JP) 2010-91732

(51) Int. Cl. A61B 8/00

(52) U.S. Cl. 600/461

(57) ABSTRACT

An ultrasound transducer or an ultrasound diagnostic apparatus that includes a body configured to transmit and receive ultrasound waves, the body having a first color, an attachment portion having a concave shape or a convex shape, the attachment portion configured to attach to a convex portion or a concave portion provided on a puncture adapter, and at least one guide mark provided on the surface of body, the at least one guide mark having a second color different from the first color.

### CROSS-REFERENCE TO RELATED APPLICATIONS

This application is based upon and claims the benefit of priority from prior Japanese Patent Application No. 2010-91732, filed Apr. 12, 2010, the entire contents of which are incorporated herein by reference.

### FIELD

Embodiments described herein relate generally to an ultrasound transducer and an ultrasound diagnostic apparatus that can attach a puncture adapter.

### BACKGROUND

An ultrasonic diagnostic apparatus is an electronic device that can generate ultrasound images. An ultrasound transducer attached to the ultrasonic diagnostic apparatus transmits ultrasonic waves to a subject, and receives reflected waves. The ultrasonic diagnostic apparatus generates ultrasound images by using the reflected waves. Conventionally, puncture diagnosis is a common practice. The puncture diagnosis harvests tissues or treats diseases by using a puncture needle. When a doctor performs the puncture diagnosis, the doctor attaches a puncture adapter to the ultrasound transducer. The doctor attaches the puncture adapter to the ultrasound transducer by connecting an attachment portion provided on the ultrasound transducer and an attachment portion provided on the puncture adapter. The doctor injects a puncture needle through the puncture adapter attached to the ultrasound transducer, and watches the ultrasound images.

Generally, when the doctor performs puncture diagnosis, the doctor folds the ultrasound transducer using a cover to prevent an infection. In Japanese published application 2009-72602, the attachment portion provided on the puncture adapter is connected to the attachment portion provided on the ultrasound transducer, after the ultrasound transducer is folded using the cover. Sometimes the cover folds over a portion of the puncture adapter.

However, the ultrasound transducer in Japanese published application 2009-72602 has an operability problem when the doctor attaches the puncture adapter to the ultrasound transducer with the cover. In particular, the attachment portion provided on the ultrasound transducer has low-visibility when the cover folds over the ultrasound transducer. Generally, the ultrasound transducer is colored white. The visibility of the attachment portion provided on the ultrasound transducer is lowered when the cover has a white color or other colors.

### BRIEF DESCRIPTION OF THE VIEWS OF THE DRAWINGS

FIGS. 1A and 1B show an appearance of an ultrasound transducer according to a first embodiment;

FIG. 2 shows a connected portion of the ultrasound transducer according to the first embodiment;

FIG. 3 shows an appearance of a puncture adapter according to the first embodiment;

FIG. 4 shows an example of the ultrasound transducer attached to the puncture adapter according to the first embodiment;

FIG. 5 shows another example of the ultrasound transducer attached to the puncture adapter according to the first embodiment;

FIGS. 6A-6C show an example of a guide mark according to the first embodiment;

FIGS. 7A-7B show another example of the guide mark according to the first embodiment;

FIG. 8 shows another example of the guide mark according to the first embodiment;

FIGS. 9A-9D show another example of the guide mark according to the first embodiment;

FIG. 10 shows an appearance of an ultrasound transducer according to a second embodiment;

FIG. 11 shows an appearance of a puncture adapter according to the second embodiment;

FIGS. 12A-12B show an example of the ultrasound transducer attached to the puncture adapter according to the second embodiment;

FIGS. 13A-13C show an example of a guide mark according to the second embodiment;

FIGS. 14A-14B show another example of the guide mark according to the second embodiment;

FIG. 15 shows another example of the guide mark according to the second embodiment;

FIGS. 16A-16D show another example of the guide mark according to the second embodiment;

FIGS. 17A-17B show an appearance of an ultrasound transducer and a puncture adapter according to a third embodiment;

FIGS. 18A-18B show an example of the ultrasound transducer attached to the puncture adapter according to the third embodiment;

FIGS. 19A-19C show an example of a guide mark according to the third embodiment;

FIGS. 20A-20B show another example of the guide mark according to the third embodiment;

FIGS. 21A-21B show another example of the guide mark according to the third embodiment;

FIGS. 22A-22B show another example of the guide mark according to the third embodiment;

FIGS. 23A-23C show an appearance of an ultrasound transducer and a puncture adapter according to a fourth embodiment;

FIGS. 24A-24B show an example of the ultrasound transducer attached to the puncture adapter according to the fourth embodiment;

FIGS. 25A-25C show an example of a guide mark according to the fourth embodiment;

FIGS. 26A-26B show another example of the guide mark according to the fourth embodiment;

FIG. 27 shows another example of the guide mark according to the fourth embodiment;

FIGS. 28A-28B show another example of the guide mark according to the fourth embodiment;

FIGS. 29A-29C show another example of the guide mark according to the fourth embodiment;

FIGS. 30A-30B show another example of the guide mark according to the fourth embodiment;

FIGS. 31A-31B show another example of the guide mark according to the fourth embodiment; and

FIGS. 32A-32B show another example of the guide mark according to the fourth embodiment.

### DETAILED DESCRIPTION

An embodiment will be described below with reference to the views of the accompanying drawings. The same reference numerals denote constituent elements having almost the same functions and arrangements, and a repetitive description will be made only when required. Note that the inventions disclosed herein are not limited to the described embodiments.

In the described embodiments, a convex portion and a concave portion are described as an attachment portion collectively. The attachment portion is provided on an ultrasound transducer, and used for attaching the ultrasound transducer and a puncture adapter. In the described embodiments, a painted pattern (for example, a figure, an arrow mark, and a line) is described as a guide mark. The guide mark is provided on the ultrasound transducer, and is used for leading the puncture adapter to an attached position.

### First Embodiment

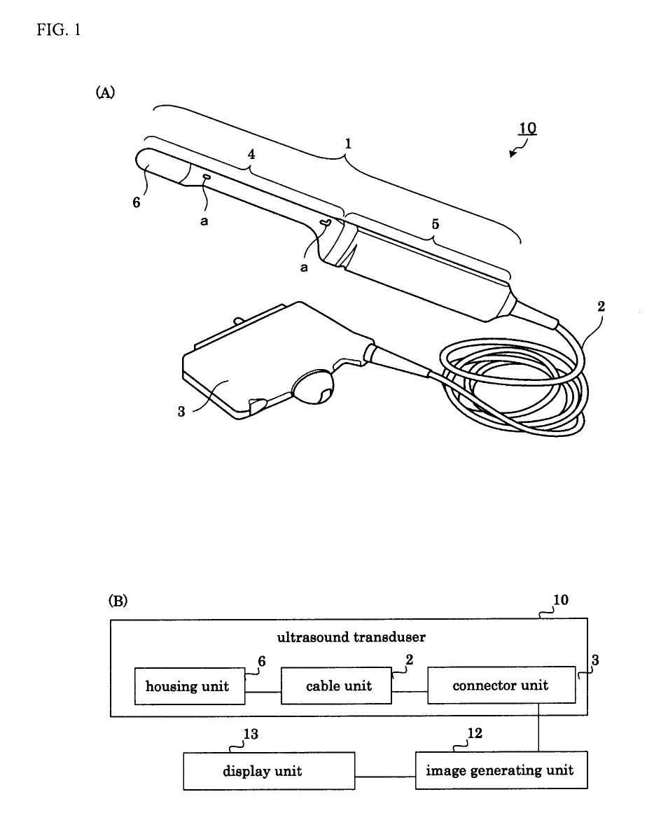

FIG. 1 (A) shows an ultrasound transducer 10 according to the first embodiment. The ultrasound transducer 10 is used for body cavity scanning, and the ultrasound transducer 10 is generally called an endocavitary transducer. As shown in FIG. 1 (A), the ultrasound transducer 10 includes a body unit 1, a cable unit 2, and a connector unit 3. The body unit 1 includes an insertion unit 4 and a grip unit 5. The insertion unit 4 is inserted into the body cavity while body cavity scanning. The grip unit 5 is held by a doctor while body cavity scanning.

FIG. 1 (B) shows a block diagram of the ultrasound transducer 10 and an ultrasound diagnostic apparatus. The body unit 1 includes a housing unit 6. The housing unit 6 is located at a tip of the insertion unit 4. The housing unit converts electronic signals into ultrasound signals, and transmits the converted ultrasound signals to a subject. The housing unit receives the reflected ultrasound signals, and converts the reflected ultrasound signals into electronic signals. The cable unit 2 transmits and receives the electronic signals between the ultrasound transducer 10 and an ultrasound diagnostic apparatus. The connector unit 3 connects the ultrasound transducer 10 and the ultrasound diagnostic apparatus electrically. The ultrasound diagnostic apparatus includes an image generating unit 12 and a display unit 13. The image generating unit 12 receives the electronic signals converted by the housing unit, and the image generating unit generates ultrasound images based on the electronic signals received. The display unit displays the ultrasound images generated by the image generating unit. Note that this embodiment describes the ultrasound transducer 10 and the ultrasound diagnostic apparatus separately, but the ultrasound diagnostic apparatus can include the ultrasound transducer 10.

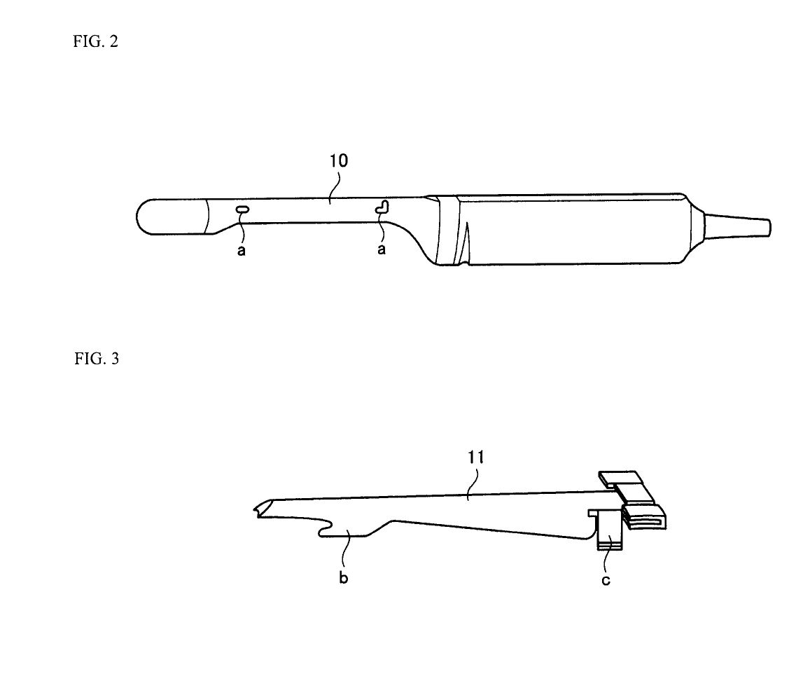

The symbol (a) in FIG. 1(A) shows a concave portion provided on the body unit 1. The concave portion (a) is used for attaching the ultrasound transducer 10 and the puncture adapter. The concave portion is described as “attachment portion” in this embodiment. In FIG. 1, two attachment portions (a) are provided on one side of the body unit 1. In this embodiment, another side of the body unit 1 also has two attachment portions (a). The body unit 1 has a total of four attachment portions (a). FIG. 2 shows a close-up view of the body unit 1 and the attachment portions (a).

FIG. 3 shows a puncture adapter 11. The convex portion (b) is provided on the puncture adapter 11, and the convex portion (b) is used for attaching the ultrasound transducer 10 and the puncture adapter 11. Note that the convex portion (b) is provided on an inner side of the puncture adapter 11. The convex portion (b) is attached to the attachment portion (a) provided on the head side of the body unit 1. In this embodiment, another inner side of the puncture adapter 11 also has the convex portion (b). Thus, puncture adapter 11 has two convex portions (b).

The convex portion (c) is provided on the puncture adapter 11, and the convex portion (c) is used for attaching the ultrasound transducer 10 and the puncture adapter 11. Note that the convex portion (c) is provided on the inner side of the puncture adapter 11. The convex portion (c) is attached to the attachment portion (a) provided on the rear side of the body unit 1. In this embodiment, another inner side of the puncture adapter 11 also has the convex portion (c). Thus, puncture adapter 11 has two convex portions (c).

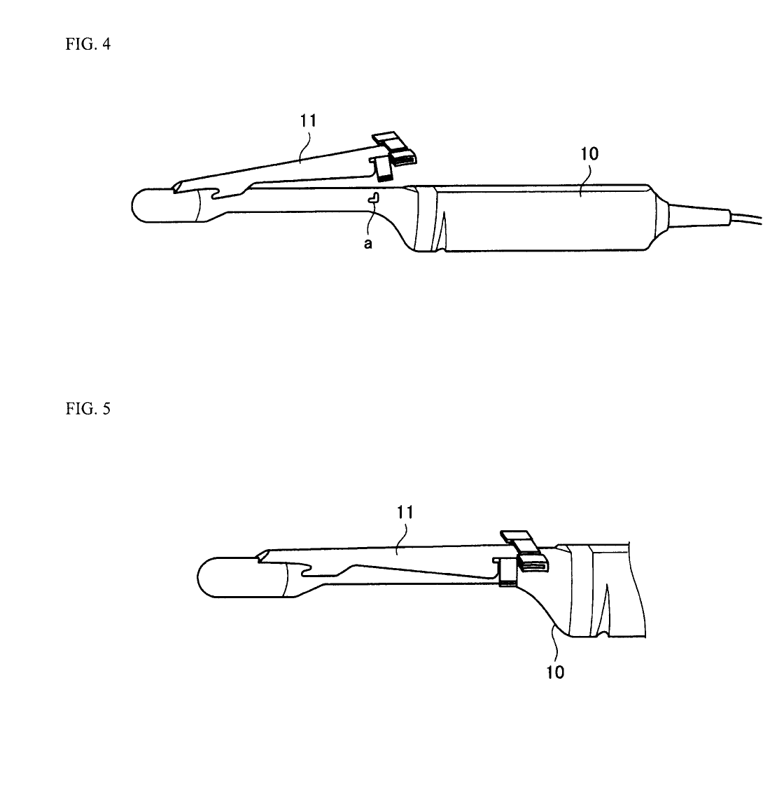

FIG. 4 and FIG. 5 show procedures of attaching the puncture adapter 11. In this embodiment, the puncture adapter 11 is attached by connecting the four attachment portions (a) to the convex portions (b) and (c), after the cover folds over the ultrasound transducer 10. As shown in FIG. 4 and FIG. 5, the convex portion (c) is attached to the attachment portion (a) provided on the rear side of the body unit 1, after the convex portion (b) is attached to the attachment portion (a) provided on the head side of the body unit 1.

Note that a conventional ultrasound transducer has an operability problem when the conventional ultrasound transducer attaches to a puncture adapter. Because when the cover folds over the conventional ultrasound adapter, it is hard to see attachment portions provided on the conventional ultrasound adapter. Generally, the conventional ultrasound transducer is colored white. The visibility of the attachment portions is lowered when the cover is colored white or other colors. In this embodiment, a guide mark that guides the doctor to attach the puncture adapter 11 is provided on the ultrasound transducer 10. The guide mark is provided on the ultrasound transducer 10 so that the doctor can check the guide mark. The guide mark improves the visibility of the attachment portions (a) and the operability of the ultrasound transducer 10.

Guide Marks in the First Embodiment

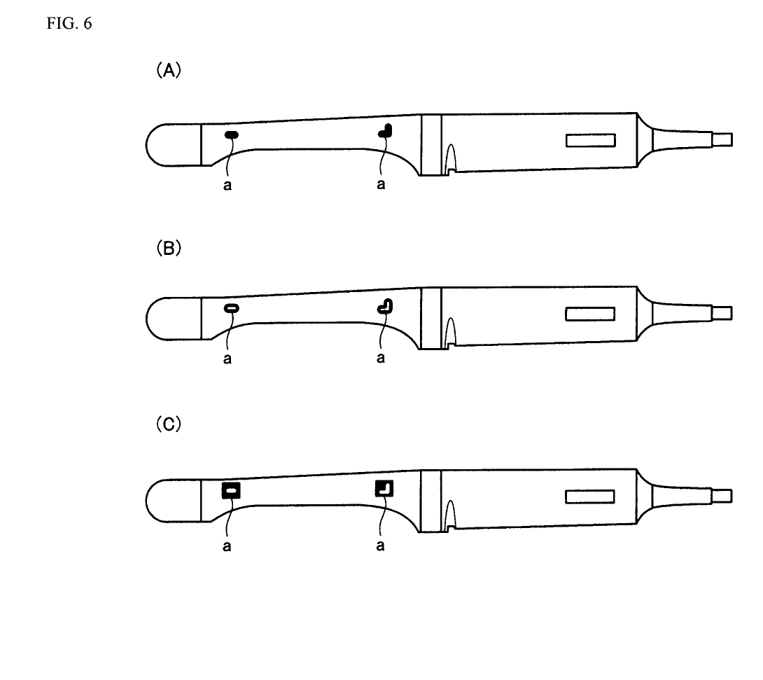

The guide marks in the first embodiment are described below. FIGS. 6 to 9 show the guide marks in the first embodiment. In FIGS. 6A-6C, the guide marks covers the attachment portions (a) or a neighborhood of the attachment portions (a). The guide marks have a different color from a color of the body unit 1. For example, in FIG. 6 (A) the guide marks covers the inner side of the attachment portion (a). As another example, in FIG. 6 (B) the guide marks cover the edge of the attachment portion (a). As another example, in FIG. 6 (C) the guide marks covers neighborhood of the attachment portion (a).

As a matter of convenience, the specific color of the guide marks is not shown in FIG. 6. The color of the guide marks can be a color that is different from the color of the body unit 1. The guide marks can be painted with two or more colors, but the color of the guide marks should not have a medical meaning. For example, red means blood, green means sterilized. A guide mark with such a color might convey misleading information. So, it is preferable that the color of the guide mark is different from a color having a medical meaning.

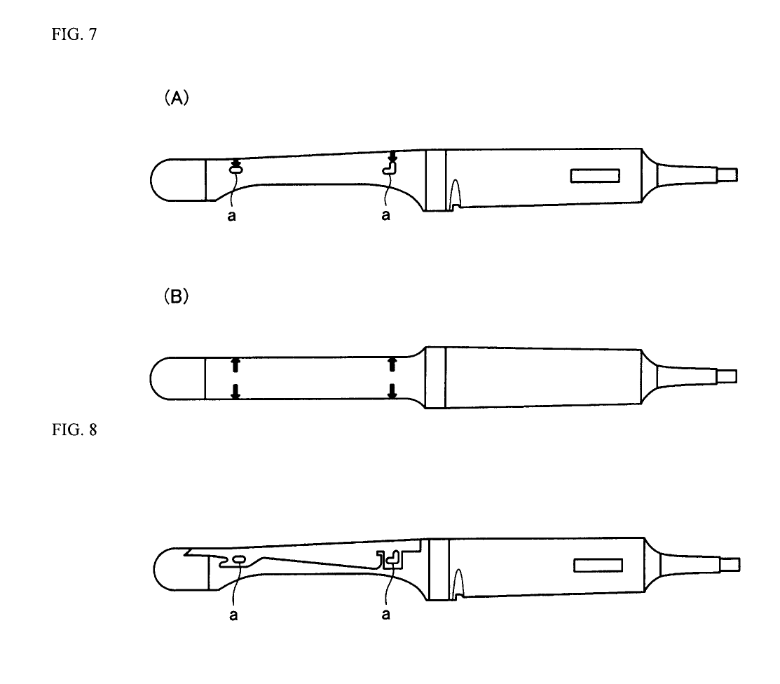

In FIGS. 7A and 7B, as another example, the guide marks cover a neighborhood of the attachment portion (a) with a color different from a color of the body unit 1. In FIG. 7 (A) the guide marks have an arrow shape that indicates the position of the attachment portions (a). The guide marks are provided on the side surface of the body unit 1. In FIG. 7 (B) the guide marks have an arrow shape and are provided on an upper side of the body unit 1. The doctor easily checks the upper side of body unit 1. The doctor can check the number of the attachment portions (a) and the positions of the attachment portions (a) by checking the upper side of body unit 1. The guide marks in FIGS. 7 (A) and (B) can be matched-up. The arrow shape of the guide marks can be frame bordered, and the arrow shapes of the guide marks can be depicted as a simple line.

In FIG. 8, as another example, the guide marks are painted as edges of a puncture adapter 11 that is to be attached to the ultrasound transducer 10. In the same way of another example, the guide marks have a color different from the color of the body unit 1. As shown in FIG. 8, the guide marks are painted as edges of the puncture adapter 11, so that the guide marks indicate the positions of the attachment portions (a).

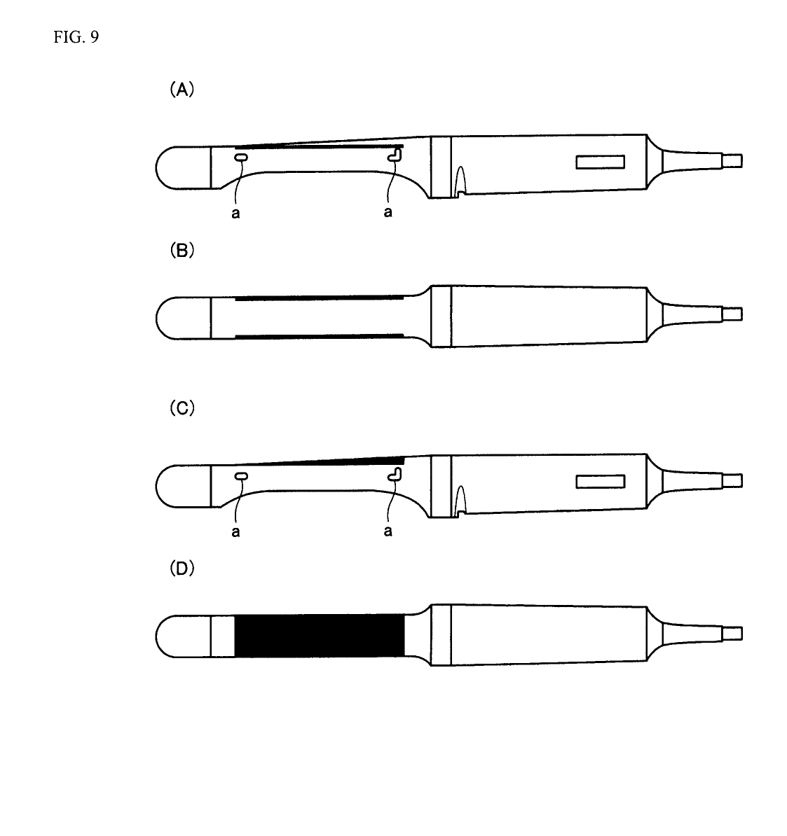

In FIGS. 9A-9D, as another example, the guide mark indicates the position of the puncture adapter 11 that is to be attached to the ultrasound transducer 10. The guide mark has a color different from the color of the body unit 1. In FIG. 9 (A), the guide mark is a horizontal line provided on the side surface of body unit 1. The doctor can check the position of the puncture adapter 11 by checking the side surface of body unit 1. In FIG. 9 (B), the guide mark is a horizontal line provided on the upper side of body unit 1. The doctor can check the position of the puncture adapter 11. The guide marks in FIGS. 9 (A) and (B) can be matched-up. The guide mark can be depicted as a narrow line or a bold line. The guide mark can be depicted as multiple lines. In FIGS. 9 (C) and (D), as another example, the guide mark can be painted on the upper side of body unit 1.

As another example, the guide mark can indicate an order of attachment. As shown in FIGS. 4 and 5, the convex portion (c) is attached to the attachment portion (a) provided on the rear side of the body unit 1, after the convex portion (b) is attached to the attachment portion (a) provided on the head side of the body unit 1. The guide mark can be depicted as numbers “1” or “2”, or can be depicted as a single line or a double line. The guide mark is painted in a neighborhood of the attachment portion (a) and the guide mark indicates the order of attachment.

As another example, the puncture adapter 11 can include multiple components. In this case, the components of the puncture adapter 11 should be attached in a predetermined order. For example, a second component of the puncture adapter 11 is attached to the attachment portion (a) provided on the rear side of the body unit 1, after the first component of the puncture adapter 11 is attached to the attachment portion (a) provided on the head of the body unit 1. As another example, a second component of the puncture adapter 11 is attached to the attachment portion (a) provided on the right side of the body unit 1, after the first component of the puncture adapter 11 is attached to the attachment portion (a) provided on the left side of the body unit 1. The guide mark is painted in a neighborhood of the attachment portion (a) and the guide mark indicates the order of attachment.

### Second Embodiment

Next, a second embodiment is described below. An ultrasound transducer 20 according to the second embodiment is used for body cavity scanning. The ultrasound transducer 20 scans a two-dimensional space. The ultrasound transducer 20 includes a body unit 1, a cable unit 2, and a connector unit 3. The body unit 1 includes the insertion unit 4 and the grip unit 5.

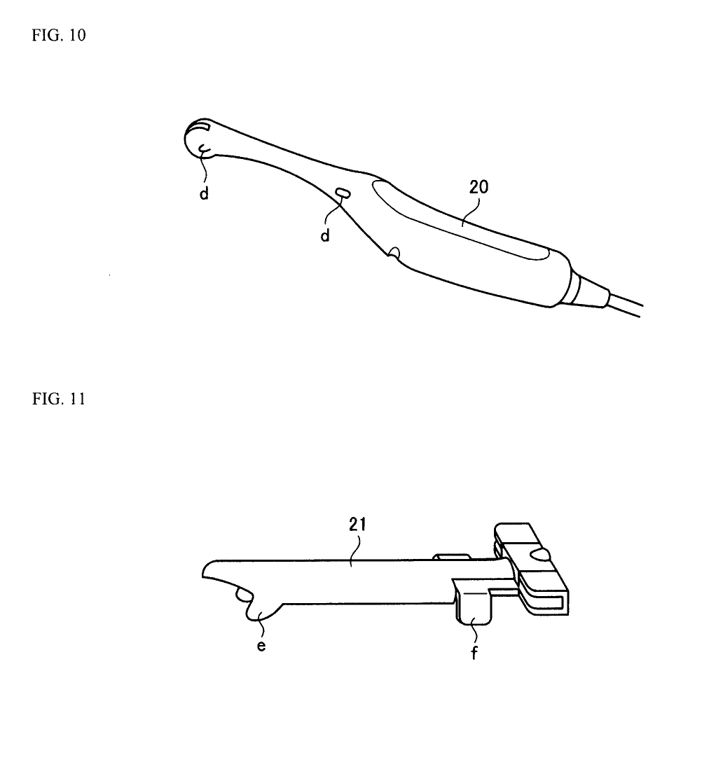

FIG. 10 shows the ultrasound transducer 20. A sign (d) in FIG. 10 shows a concave portion provided on the body unit 1. The concave portion (d) is used for attaching the ultrasound transducer 20 and the puncture adapter 21 described below. The concave portion is described as an “attachment portion” in this embodiment. In FIG. 10, two attachment portions (d) are provided on one side of the body unit 1. In this embodiment, another side of the body unit 1 also has the two attachment portions (d). The body unit 1 has a total of four attachment portions (d).

FIG. 11 shows an appearance of the puncture adapter 21. The convex portion (e) is provided on the puncture adapter 21, and the convex portion (e) is used for attaching the ultrasound transducer 20 and the puncture adapter 21. Note that the convex portion (e) is provided on an inner side of the puncture adapter 21. The convex portion (e) is attached to the attachment portion (d) provided on the head side of the body unit 1. In this embodiment, another inner side of the puncture adapter 21 also has the convex portion (e). Thus, the puncture adapter 21 has the two convex portions (e).

The convex portion (f) is provided on the puncture adapter 21, and the convex portion (f) is used for attaching the ultrasound transducer 20 and the puncture adapter 21. Note that the convex portion (f) is provided on the inner side of the puncture adapter 21. The convex portion (f) is attached to the attachment portion (d) provided on the rear side of the body unit 1. In this embodiment, another inner side of the puncture adapter 21 also has the convex portion (f). Thus, the puncture adapter 21 has two convex portions (f).

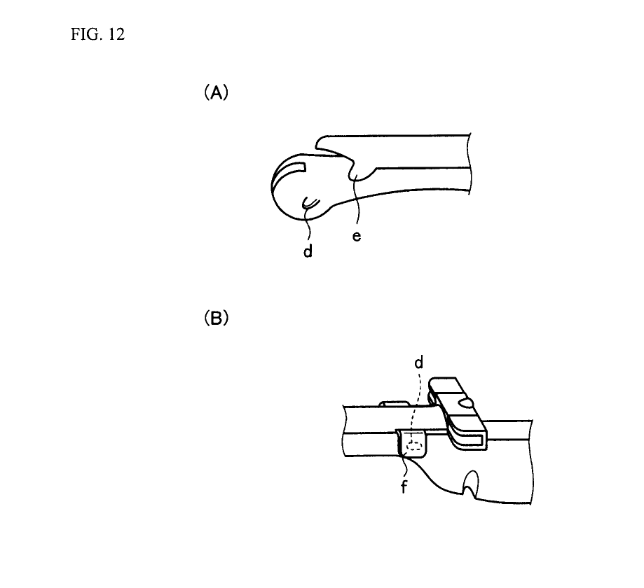

FIGS. 12A and 12B shows procedures of attaching the puncture adapter 21. In this embodiment, the puncture adapter 21 is attached by connecting the four attachment portions (d) to the convex portions (e) and (f), after the cover folds over the ultrasound transducer 20. As shown in FIGS. 12A and 12B, the convex portions (f) are attached to the attachment portions (d) provided on the rear side of the body unit 1, after the convex portions (e) are attached to the attachment portion (d) provided on the head side of the body unit 1.

Guide Marks in the Second Embodiment

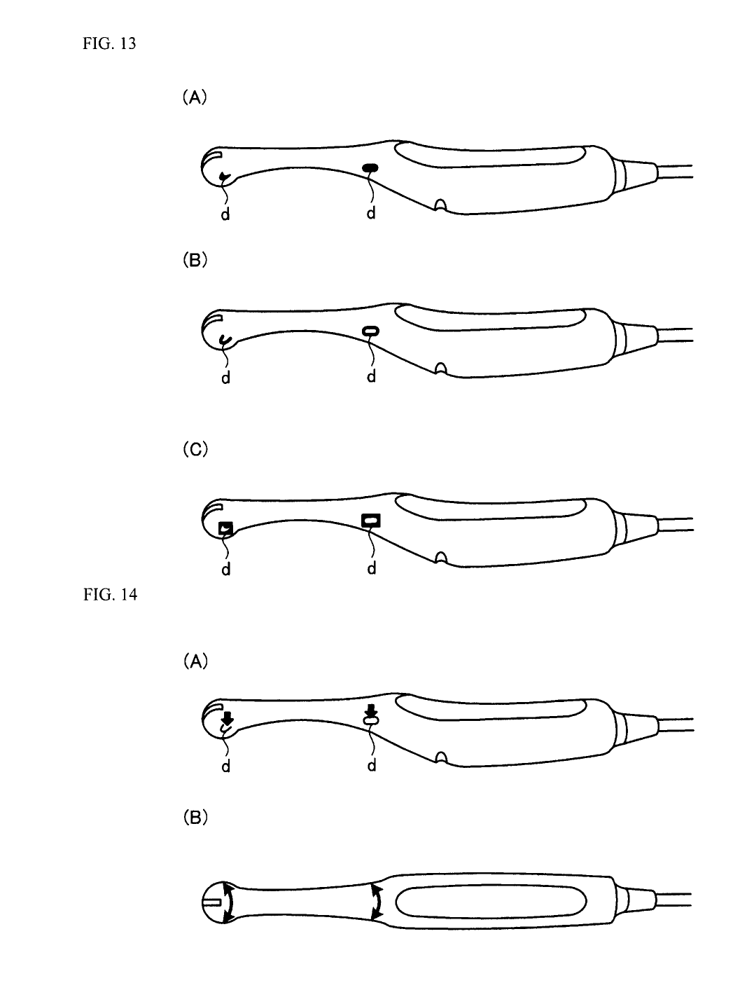

The guide marks in the second embodiment are described below. FIGS. 13 to 16 shows the guide marks in the second embodiment. In FIGS. 13A-13C, the guide marks cover the attachment portions (d) or a neighborhood of the attachment portions (d). The guide marks have a different color from a color of the body unit 1. For example, in FIG. 13 (A) the guide marks cover an inner side of the attachment portion (d). As another example, in FIG. 13 (B) the guide marks cover an edge of the attachment portions (d). As another example, in FIG. 13 (C) the guide marks cover a neighborhood of the attachment portions (d).

As a matter of convenience, the specific color of the guide mark is not shown in FIG. 13. The color of the guide mark can be a color that is different from the color of the body unit 1. The guide marks can be painted with two or more colors, but the color of the guide marks should not have a medical meaning.

In FIGS. 14A and 14B, as another example, the guide marks cover a neighborhood of the attachment portion (d) with a color different from the body unit 1. In FIG. 14 (A) the guide mark has an arrow shape that indicates a position of the attachment portions (d). The guide marks are provided on a side surface of the attachment portions (d). In FIG. 14 (B) the guide marks have an arrow shape and are provided on an upper side of the body unit 1. The doctor easily checks the upper side of body unit 1. The doctor can check the number of the attachment portions (d) and the positions of the attachment portions (d) by checking the upper side of body unit 1. The guide marks in FIGS. 14 (A) and (B) can be matched-up. The arrow shape of the guide marks can be frame bordered, and the arrow shape of the guide marks can be depicted as a simple line.

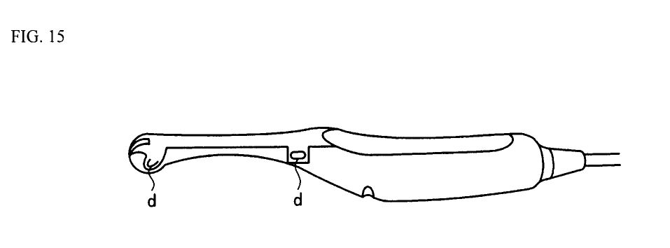

In FIG. 15, as another example, the guide marks are painted as edges of the puncture adapter 21 that is to be attached to the ultrasound transducer 20. In the same way of another example, the guide marks have a color different from the color of the body unit 1. As shown in FIG. 15, the guide marks are painted as edges of the puncture adapter 21, so that the guide marks indicate the position of the attachment portions (d).

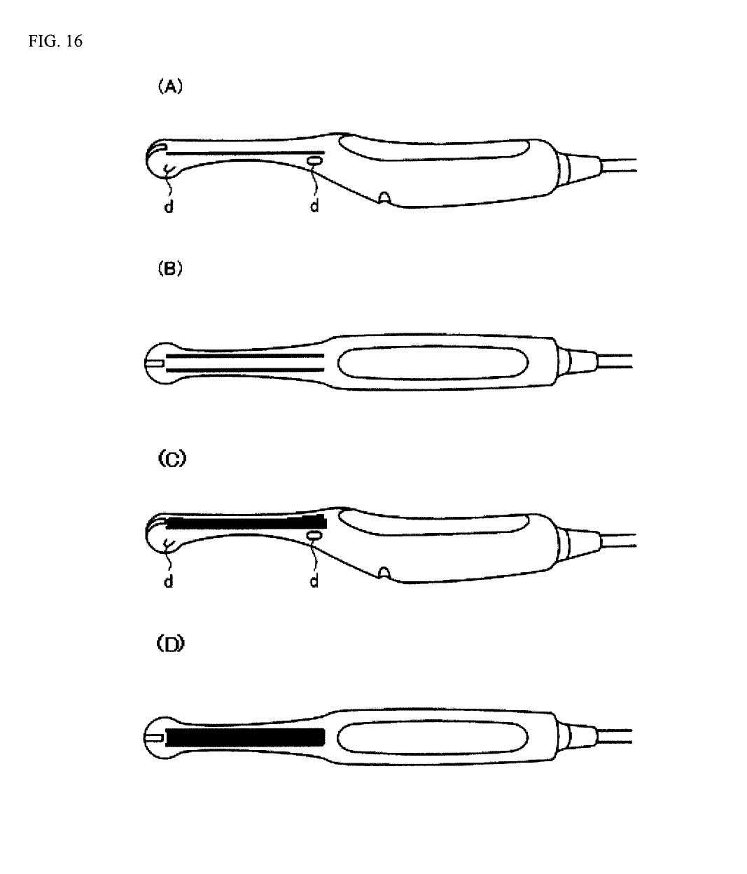

In FIGS. 16A and 16B, as another example, the guide mark indicates a position of the puncture adapter 21 that is to be attached to the ultrasound transducer 20. The guide mark has a color different from a color of the body unit 1. In FIG. 16 (A), the guide mark is a horizontal line provided on the side surface of body unit 1. The doctor can check the position of the puncture adapter 21 by checking the side surface of body unit 1. In FIG. 16 (B), the guide mark is a horizontal line provided on the upper side of body unit 1. The doctor can check the position of the puncture adapter 21. The guide marks in FIGS. 16 (A) and (B) can be matched-up. The guide marks can be depicted as a narrow line or a bold line. The guide mark can be depicted as multiple lines. In FIGS. 16 (C) and (D), as another example, the guide mark can be painted on the upper side of body unit 1.

As another example, the guide marks can indicate an order of attachment. As shown in FIG. 12, the convex portions (f) are attached to the attachment portions (d) provided on the rear side of the body unit 1, after the convex portions (e) are attached to the attachment portions (d) provided on the head side of the body unit 1. The guide marks can be depicted as a number “1” or “2”, or can be depicted as a single line or a double line. The guide marks are painted in a neighborhood of the attachment portions (g) and the guide marks indicated the order of attachment.

As another example, the puncture adapter 21 can have multiple components. In this case, the components of the puncture adapter 21 should attach in a predetermined order. For example, a second component of the puncture adapter 21 is attached to the attachment portion (d) provided on the rear side of the body unit 1, after the first component of the puncture adapter 21 is attached to the attachment portion (d) provided on the head of the body unit 1. As another example, a second component of the puncture adapter 21 is attached to the attachment portion (d) provided on the right side of the body unit 1, after the first component of the puncture adapter 21 is attached to the attachment portion (d) provided on the left side of the body unit 1. The guide mark is painted in a neighborhood of the attachment portion (d) and the guide marks indicate the order of attachment.

### Third Embodiment

Next, a third embodiment is described below. An ultrasound transducer 30 according to the third embodiment is used for body surface scanning. The ultrasound transducer 30 includes a body unit 1, a cable unit 2, and a connector unit 3. The body unit 1 includes an insertion unit 4 and the grip unit 5.

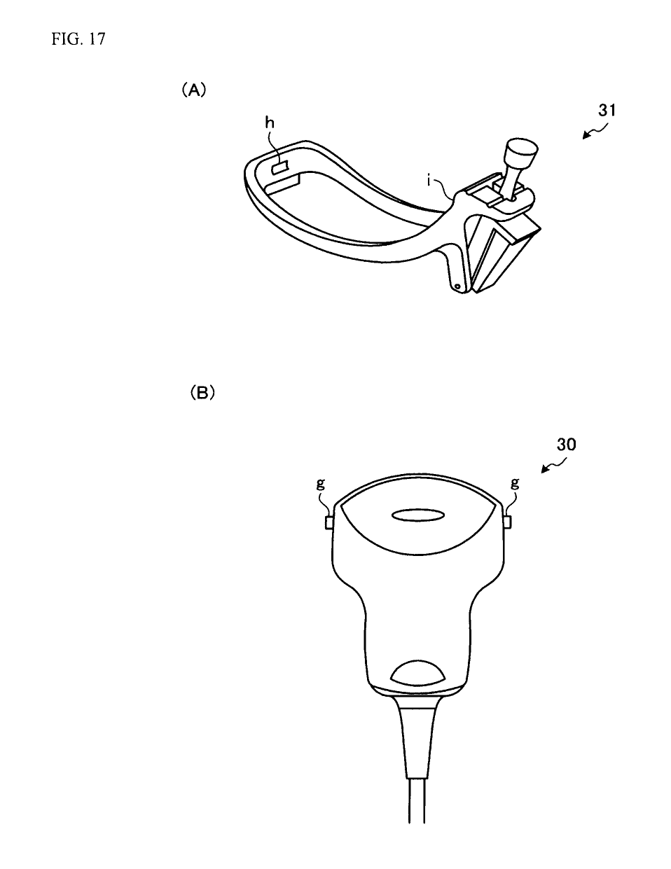

FIGS. 17A and 17B show the ultrasound transducer 30 and a puncture adapter 31. FIG. 17 (A) shows the puncture adapter 31, and FIG. 17 (B) shows the ultrasound transducer 30. A sign (g) in FIG. 17 (B) shows convex portions provided on the body unit 1. The convex portions (g) are used for attaching the ultrasound transducer 30 and the puncture adapter 31 described below. The convex portion is described as “attachment portion” in this embodiment. In FIG. 17 (B), the two attachment portions (g) are provided on both sides of the body unit 1. In this embodiment, the body unit 1 has two attachment portions (g).

FIG. 17 (A) shows the puncture adapter 31. The concave portions (h) and (l) are provided on the puncture adapter 31, and the concave portions (h) and (l) are used for attaching the ultrasound transducer 30 and the puncture adapter 31. Note that the concave portions (h) and (l) are provided on an inner side of the puncture adapter 31.

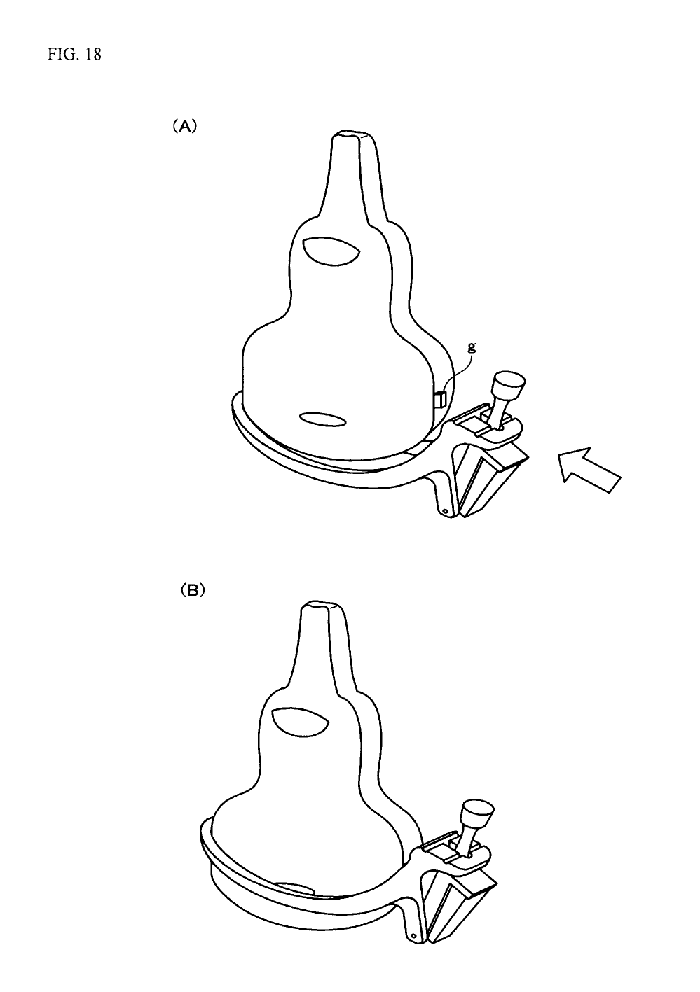

FIG. 18 shows procedures of attaching the puncture adapter 31. In this embodiment, the puncture adapter 31 is attached by connecting the two attachment portions (g) to the concave portion (h) and (l), after the cover folds over the ultrasound transducer 30. As shown in FIG. 18, the concave portion (h) is attached to the attachment portion (g) provided on the right side of the body unit 1, after the concave portion (h) is attached to the attachment portion (g) provided on the left side of the body unit 1.

Guide Marks in the Third Embodiment

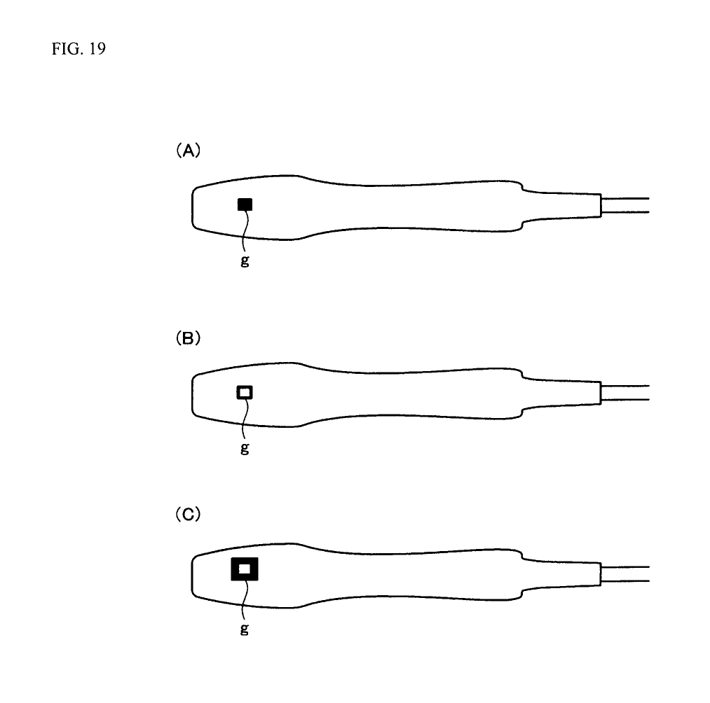

The guide marks in the third embodiment are described below. FIGS. 19 to 22 shows the guide marks in the third embodiment. FIGS. 19A-19C shows the ultrasound transducer 30 as viewed from the arrow in FIG. 18 (A). As shown in FIGS. 19A-19C, the guide marks cover the attachment portion (g) or a neighborhood of the attachment portion (g). The guide mark has a different color from a color of the body unit 1. For example, in FIG. 19 (A), the guide mark covers the surface of the attachment portion (g). As another example, in FIG. 19 (B) the guide mark covers an edge of the attachment portion (g). As another example, in FIG. 19 (C), the guide mark covers a neighborhood of the attachment portion (g).

As a matter of convenience, the specific color of the guide mark is not shown in FIGS. 19A-19C. The color of the guide mark can be a color that is different from the color of the body unit 1. The guide mark can be painted with two or more colors, but the color of the guide mark should not have a medical meaning.

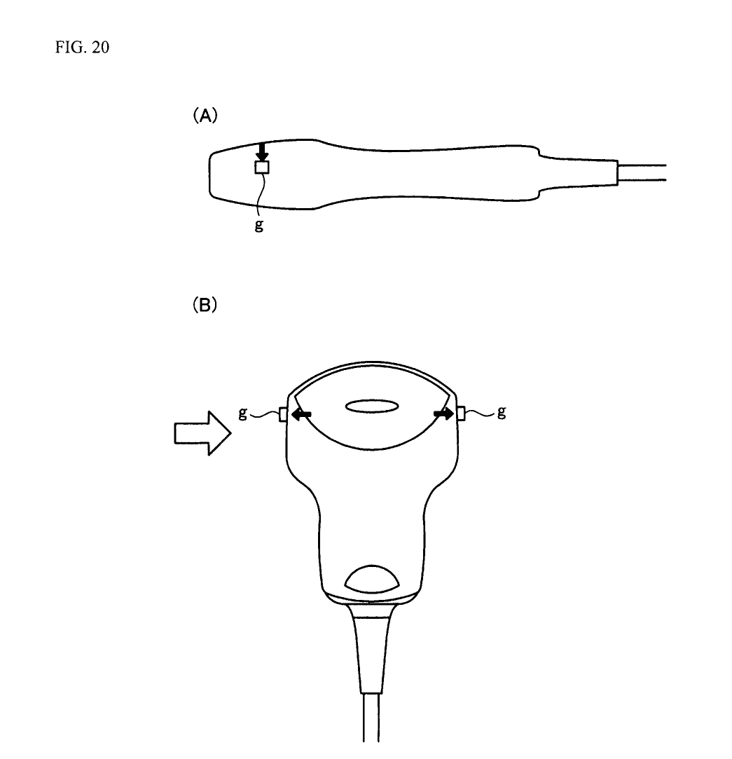

In FIGS. 20A-20B, as another example, the guide mark is painted in a neighborhood of the attachment portion (g) with a color different from the body unit 1. In FIG. 20 (A) the guide mark has an arrow shape that indicates a position of the attachment portion (d). The guide mark is provided on the side surface of the body unit 1. Note that FIG. 20 (A) shows the ultrasound transducer 30 as viewed from an arrow in FIG. 20 (B). In FIG. 20 (B), the guide mark has an arrow shape and is provided on the upper side of body unit 1. The doctor easily checks the upper side of body unit 1. The doctor can check a number of the attachment portion (g) and a position of the attachment portion (g) by checking the upper side of body unit 1. The guide marks in FIGS. 20 (A) and (B) can be matched-up. The arrow shape of a guide mark can be frame bordered, and the arrow shape of a guide mark can be depicted as a simple line.

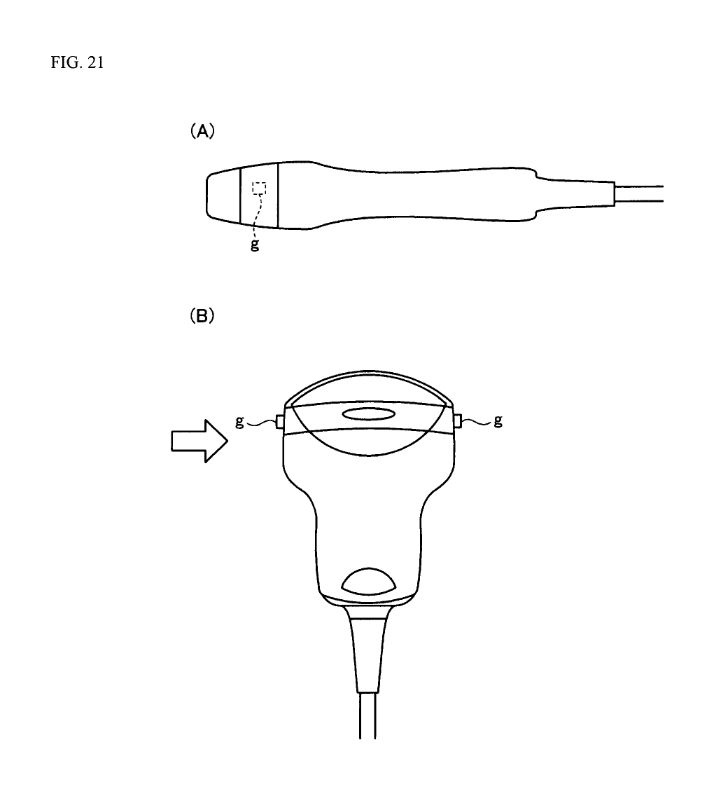

In FIGS. 21A and 21B, as another example, the guide mark is painted as edges of the puncture adapter 31 that is attached to the ultrasound transducer 30. FIG. 21 (A) shows the guide mark painted on a side surface of the body unit 1. As shown in FIG. 15, the guide mark is painted as edges of the puncture adapter 21, so that the guide mark indicates the position of the attachment portion (g). Note that FIG. 21 (A) shows the ultrasound transducer 30 as viewed from an arrow in FIG. 20 (B). In FIG. 20 (B) the guide mark is provided on the upper side of body unit 1.

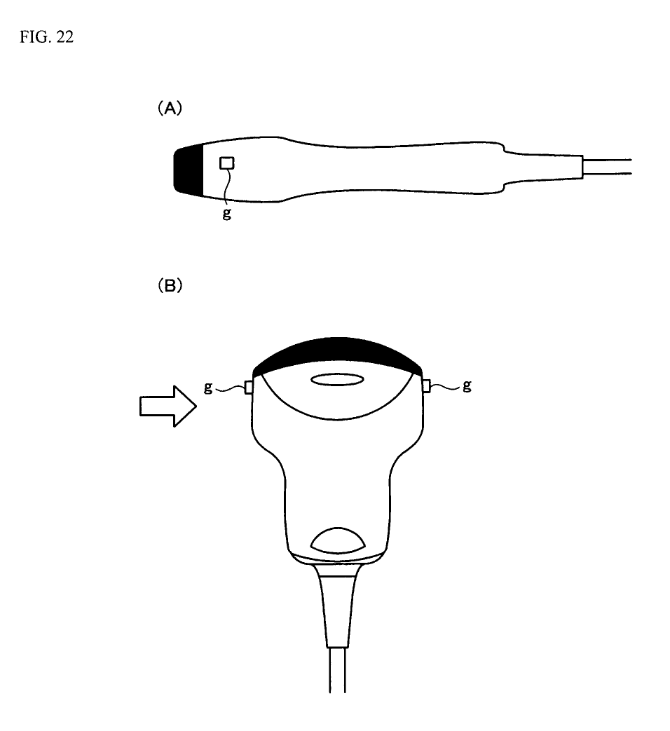

In FIGS. 22A and 22B, as another example, the guide mark is painted on the area of puncture adapter 31 that is attached to the ultrasound transducer 30. For example, FIG. 22 (A) shows the guide mark painted on the head side of ultrasound transducer 30. The doctor can check the position of the puncture adapter 31 by checking the side surface of body unit 1. Note that FIG. 22 (A) shows the ultrasound transducer 30 as viewed from the arrow in FIG. 22 (B). In FIG. 22 (B) the guide mark is provided on the upper side of body unit 1. The guide marks in FIGS. 22 (A) and (B) can be matched-up. The guide mark can be depicted as the edge of puncture adapter 31 that is attached to the ultrasound transducer 30.

As another example, the guide mark can indicate an order of attachment. As shown in FIGS. 18A and 18B, the convex portion (l) is attached to the attachment portion (g) provided on the left side of the body unit 1, after the convex portion (h) is attached to the attachment portion (g) provided on the right side of the body unit 1. The guide mark can be depicted as a number “1” or “2”, or can be depicted as a single line or a double line. The guide mark is painted in a neighborhood of the attachment portion (g) and the guide mark indicates the order of attachment.

As another example, the puncture adapter 31 can include multiple components. In this case, the components of puncture adapter 31 should attach in predetermined order. For example, a second component of the puncture adapter 31 is attached to the attachment portion (g) provided on the left side of the body unit 1, after the first component of puncture adapter 21 is attached to the attachment portion (g) provided on the right of the body unit 1. The guide marks are painted in a neighborhood of the attachment portion (d) and the guide marks indicate the order of attachment.

### Fourth Embodiment

Next, the fourth embodiment is described below. An ultrasound transducers 40, 45 according to the fourth embodiment are used for body surface scanning. The ultrasound transducers 40, 45 include a body unit 1, a cable unit 2, and a connector unit 3. The body unit 1 includes an insertion unit 4 and an grip unit 5.

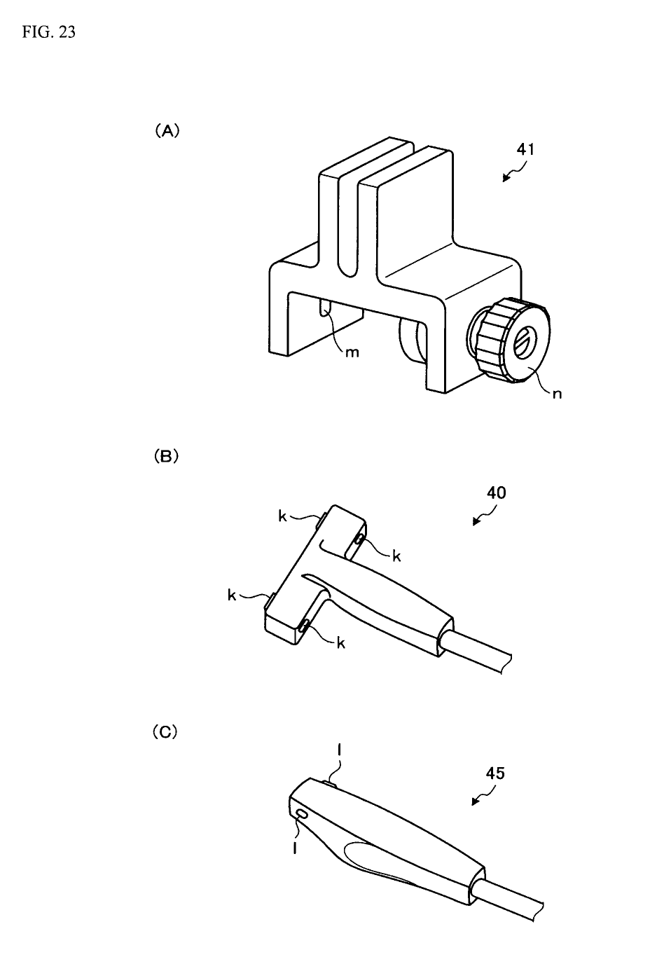

FIGS. 23A-23C show the ultrasound transducers 40, 45 and a puncture adapter 41. FIG. 23 (A) shows the puncture adapter 41, and FIG. 23 (B) shows the ultrasound transducer 40, and FIG. 23 (C) shows the ultrasound transducer 45. The symbol (k) in FIG. 23 (B) shows a convex portion provided on the body unit 1. The convex portion (k) is used for attaching the ultrasound transducer 40 and the puncture adapter 41 described below. The convex portion is described as an “attachment portion” in this embodiment. In FIG. 23 (B), two attachment portions (k) are provided on each side of the body unit 1 each. In this embodiment, the body unit 1 has a total of four attachment portions (k).

FIG. 23 (C) shows the ultrasound transducer 45. The symbol (l) in FIG. 23 (C) shows a convex portion provided on the body unit 1. The convex portion (l) is used for attaching the ultrasound transducer 45 and the puncture adapter 41 described below. The convex portion is described as an “attachment portion” in this embodiment. In FIG. 23 (C), two attachment portions (l) are provided on each side of the body unit 1. In this embodiment, the body unit 1 has a total of two attachment portions (l).

FIG. 23 (A) shows the puncture adapter 41. A concave portion (m) is provided on the puncture adapter 41, and the concave portion (m) is used for attaching the ultrasound transducer 40 or the ultrasound and transducer 45 and the puncture adapter 41. The concave portion (m) is provided on an inner side of the puncture adapter 41. The symbol (n) in FIG. 23 (A) shows a clincher used to steady the puncture adapter 41.

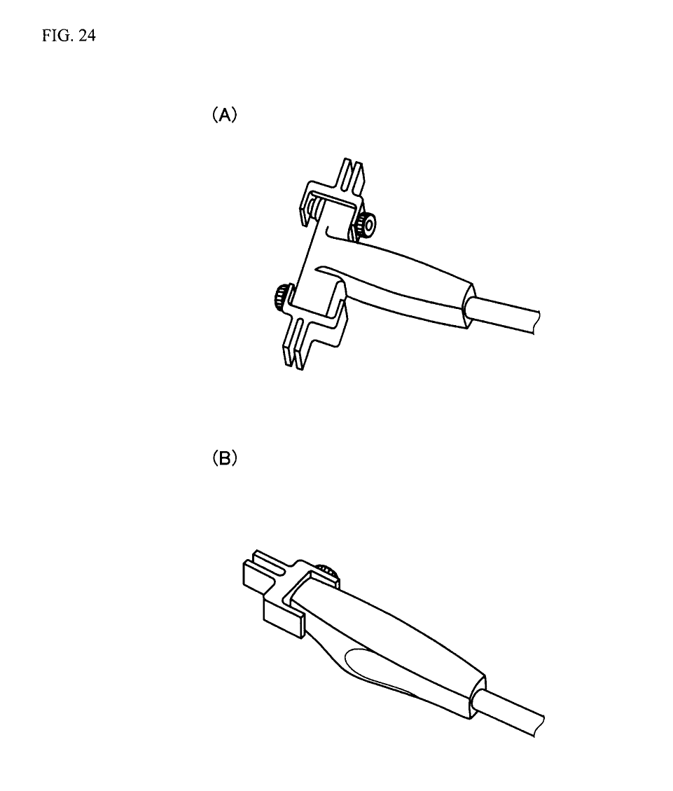

FIGS. 24A and 24B show procedures of attaching the puncture adapter 41. In this embodiment, the puncture adapter 41 is attached by connecting the attachment portions (k) or (l) and the concave portion (m), after the cover folds over the ultrasound transducer 40 or 45. As shown in FIG. 24 (A), the ultrasound transducer 40 can attach to the two-puncture adapter 41. For example, the puncture adapter 41 is attached by connecting the concave portion (m) and the attachment portion (k). Then clincher (n) clinches between the inner side of puncture adapter 41 and the attachment portion (k), and the clincher (n) fixes the position of puncture adapter 41. The ultrasound transducer 40 can attach to the two-puncture adapter 41.

As shown in FIG. 24 (B), the ultrasound transducer 45 can attach to the one-puncture adapter 41. For example, the puncture adapter 41 is attached by connecting the concave portion (m) and the attachment portion (l). Then clincher (n) clinches between the inner side of puncture adapter 41 and the attachment portion (l), and the clincher (n) fixes the position of puncture adapter 41.

Guide Marks in the Fourth Embodiment

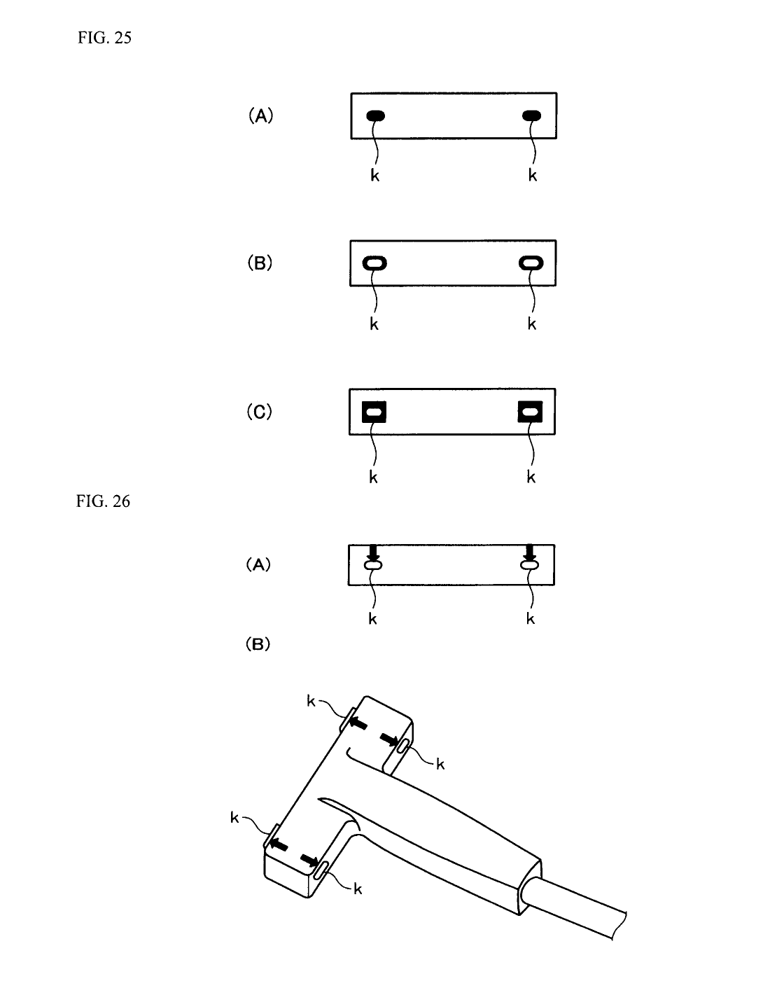

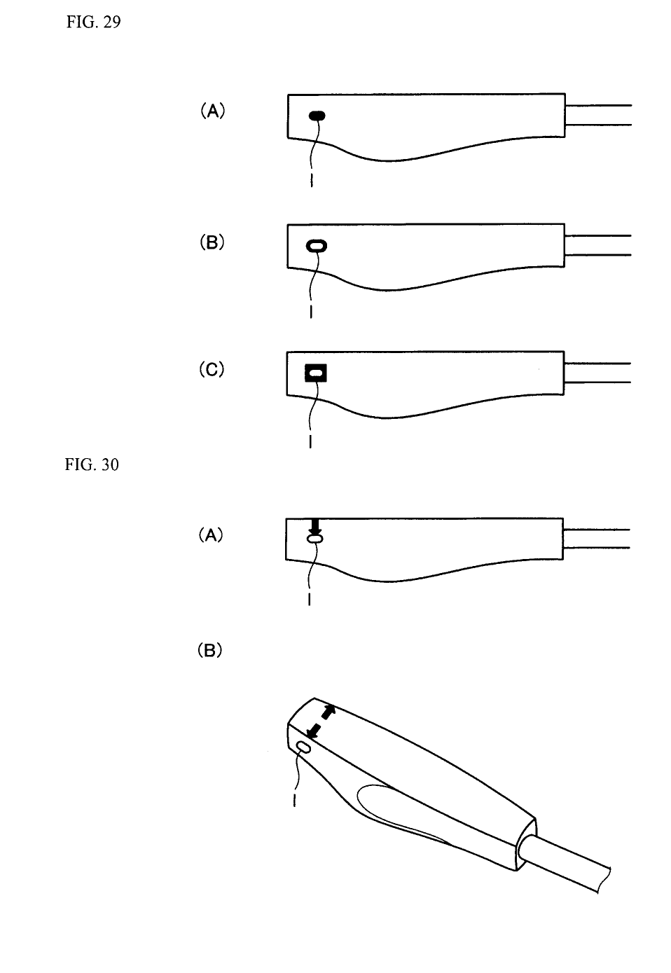

The guide marks in the fourth embodiment are described below. FIGS. 25 to 32 show the guide marks in the fourth embodiment. FIGS. 25 to 28 show the guide marks provided on the ultrasound transducer 40, and FIGS. 29 to 32 show the guide marks provided on the ultrasound transducer 45. As shown in FIGS. 25A-25C, the guide marks cover the attachment portion (k) or a neighborhood of the attachment portion (k). Each guide mark has a different color from a color of the body unit 1. For example, in FIG. 25 (A) the guide marks cover the surface of the attachment portion (k). As another example, in FIG. 25 (B) the guide marks cover an edge of the attachment portion (k). As another example, in FIG. 25 (C) the guide marks cover a neighborhood of the attachment portion (k).

As a matter of convenience, a specific color of the guide marks is not shown in FIG. 25. The color of the guide marks can be different from the color of the body unit 1. The guide marks can be painted with two or more colors, but the color of a guide mark should not have a medical meaning.

In FIGS. 26A and 26B, as another example, the guide marks cover a neighborhood of the attachment portion (k) with a color different from the body unit 1. In FIG. 26 (A) the guide mark has an arrow shape that indicates a position of the attachment portion (k). The guide mark is provided on the side surface of the body unit 1. In FIG. 26 (B), the guide mark has an arrow shape and is provided on the upper side of body unit 1. The doctor easily checks the upper side of body unit 1. The doctor can check a number of the attachment portion (k) and a position of the attachment portion (k) by checking the upper side of body unit 1. The guide marks in FIGS. 20 (A) and (B) can be matched-up. The arrow shape of the guide marks can be frame bordered, and the arrow shape of the guide marks can be depicted as a simple line.

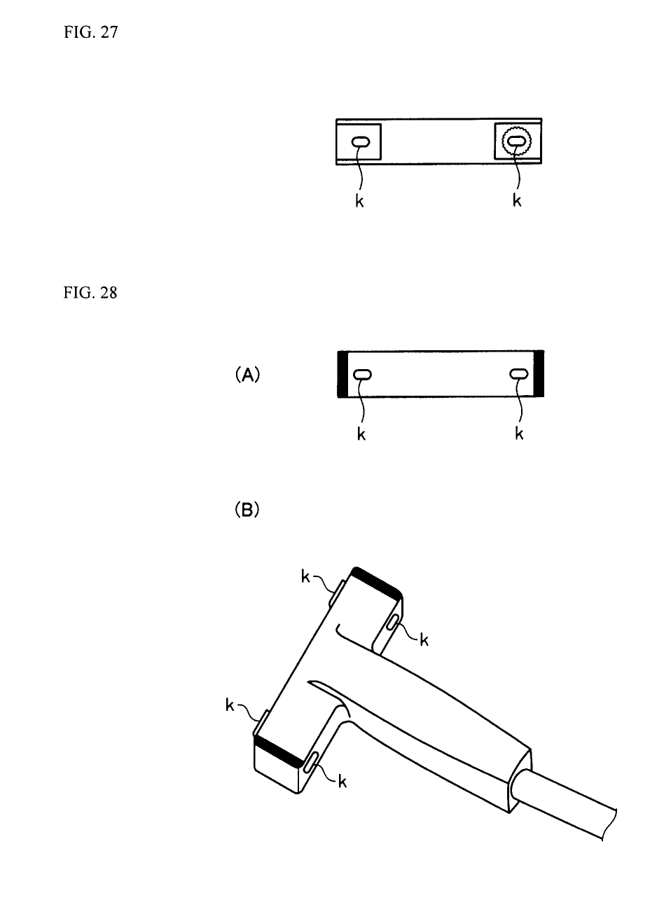

In FIG. 27, as another example, the guide mark is painted as edges of the puncture adapter 41 that is attached to the ultrasound transducer 40. As shown in FIG. 27, the guide mark is painted as edges of the puncture adapter 21, so that the guide mark indicates the position of the attachment portion (k). The guide mark is also painted as edges of the clincher (n) so that the doctor can check the direction in which the puncture adapter 41 attached.

In FIG. 28, as another example, the guide mark is painted on the area of the puncture adapter 41 that is attached to the ultrasound transducer 40. For example, FIG. 28 (A) shows the guide mark covering the side surface of the ultrasound transducer 40. The doctor can check the position of the puncture adapter 41 by checking the side surface of body unit 1. In FIG. 28 (B) the guide mark is provided on the upper side of body unit 1. The doctor can check that the ultrasound transducer 40 can attach to the puncture adapter on both sides when the doctor checks the upper side of body unit 1. The guide marks in FIGS. 22 (A) and (B) can be matched-up. The guide marks can be depicted as the edge of the puncture adapter 41 that is attached to the ultrasound transducer 40.

FIGS. 29 to 32 shows the guide marks provided on the ultrasound transducer 45. As shown in FIGS. 29A-29C, the guide marks cover the attachment portion (l) or a neighborhood of the attachment portion (l). The guide marks have a different color from a color of the body unit 1. For example, in FIG. 29 (A) the guide mark covers the surface of the attachment portion (l). As another example, in FIG. 29 (B) the guide marks cover an edge of the attachment portion (l). As another example, in FIG. 29 (C) the guide marks cover a neighborhood of the attachment portion (l).

As a matter of convenience, the specific color of the guide marks is not shown in FIGS. 29A-29C. The color of the guide marks can be different from the color of the body unit 1. The guide marks can be painted with two or more colors, but the color of the guide marks should not have a medical meaning.

In FIGS. 30A and 30B, as another example, the guide marks cover a neighborhood of the attachment portion (l) with a color different from the body unit 1. In FIG. 30 (A) the guide mark has an arrow shape that indicates the position of the attachment portion (l). The guide mark is provided on the side surface of the body unit 1. In FIG. 30 (B), the guide marks have an arrow shape and are provided on the upper side of body unit 1. The doctor easily checks the upper side of body unit 1. The doctor can check a number of the attachment portion (l) and the position of the attachment portion (l) by checking the upper side of body unit 1. The guide marks in FIGS. 30 (A) and (B) can be matched-up. The arrow shape of guide marks can be frame bordered, and the arrow shape of the guide marks can be depicted as a simple line.

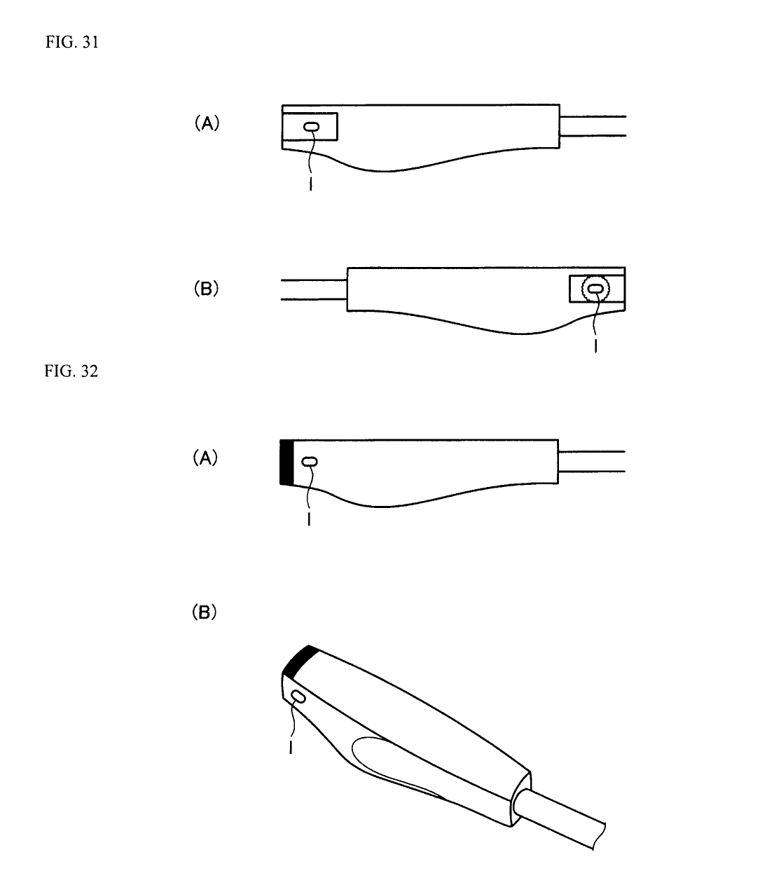

In FIGS. 31A-31B, as another example, the guide mark is painted as edges of the puncture adapter 41 that is attached to the ultrasound transducer 45. As shown in FIGS. 31A-31B, the guide mark is painted as edges of the puncture adapter 41, so that the guide mark indicates the position of the attachment portion (l). The guide mark is also painted as edges of the clincher (n) so that the doctor can check the direction of the puncture adapter 41 attached.

In FIGS. 32A-32B, as another example, the guide mark covers the area of puncture adapter 41 that is attached to the ultrasound transducer 45. For example, FIG. 32 (A) shows the guide mark covering the side surface of ultrasound transducer 45. The doctor can check the position of the puncture adapter 41 by checking the side surface of body unit 1. In FIG. 32 (B) the guide mark is provided on the upper side of body unit 1. The doctor can check that the ultrasound transducer 45 can attach to the puncture adapter on both sides when the doctor checks the upper side of body unit 1. The guide marks in FIGS. 32 (A) and (B) can be matched-up. The guide marks can be depicted as the edge of the puncture adapter 41 that is attached to the ultrasound transducer 45.

As another example, the guide marks can indicate an order of attachment. As shown in FIGS. 24A and 24B, the convex portion (l) is attached to the attachment portion (k) provided on the left side of the body unit 1, after the convex portion (l) is attached to the attachment portion (k) provided on the right side of the body unit 1. The guide marks can be depicted as a number “1” or “2”, or can be depicted as a single line or double line. The guide marks cover a neighborhood of the attachment portion (k) and the guide marks indicate the order of attachment.

As another example, the puncture adapter 41 can include multiple components. In this case, the components of puncture adapter 41 should attach in predetermined order. For example, the second component of puncture adapter 41 is attached to the attachment portion (k) provided on the left side of the body unit 1, after the first component of the puncture adapter 41 is attached to the attachment portion (k) provided on the right of the body unit 1. The guide marks cover a neighborhood of the attachment portion (k) and the guide marks indicate the order of attachment.

Heretofore the first to fourth embodiments are described, but these embodiments are not intended to limit the scope of the claimed inventions. For example, types of the ultrasound transducer are not limited to the disclosed embodiments. The guide marks can be adapted to any type of ultrasound transducer attached to the puncture adapter. Further, the guide marks are not limited to the disclosed embodiments. An area of coverage, the shapes of the guide marks, and the method of indicating can be changed. Indeed, the novel components and systems described herein may be embodied in a variety of other forms. Furthermore, various omissions, substitutions, and changes in the form of the components and systems described herein may be made without departing from the spirit of the claimed inventions. The accompanying claims and their equivalents are intended to cover such forms or modifications as would fall within the scope and spirit of the claimed inventions.

1. An ultrasound transducer, comprising:
a body configured to transmit and receive ultrasound waves, the body having a first color;
an attachment portion having a concave shape or a convex shape, the attachment portion configured to attach to a convex portion or a concave portion provided on a puncture adapter; and
at least one guide mark provided on a surface of the body, the at least one guide mark having a second color different from the first color.

2. The ultrasound transducer of claim 1, wherein
the at least one guide mark is colored on the concave shape or the convex shape of the attachment portion.

3. The ultrasound transducer of claim 1, further comprising:
an attachment surface provided on the body, the attachment surface configured to attach to the puncture adapter,
wherein the at least one guide mark is provided on the attachment surface, the at least one guide mark indicating a position of the attachment portion.

4. The ultrasound transducer of claim 3, wherein
the at least one guide mark has an arrow shape, the arrow shape of the at least one guide mark indicating the position of the attachment portion.

5. The ultrasound transducer of claim 1, further comprising:
an attachment surface provided on the body, the attachment surface configured to attach to the puncture adapter,
wherein the at least one guide mark is provided on the attachment surface, the at least one guide mark indicating a position of the attachment surface.

6. The ultrasound transducer of claim 5, wherein
the at least one guide mark is colored on an edge of the attachment surface or an inside edge of the attachment surface.

7. The ultrasound transducer of claim 1, wherein
more than one attachment portion and more than one guide mark are provided on the body, each guide mark depicting a number, the number indicating an order of attachment of the attachment portion, or the number indicating an order of attachment of multiple parts of the puncture adapter.

8. The ultrasound transducer of claim 1, further comprising:
an attachment surface provided on the body, the attachment surface configured to attach to the puncture adapter,
wherein the body comprises an insertion unit configured to insert into a cavity, and a grip unit configured to be held by an operator; and
the at least one guide mark is provided on a surface of the insertion unit.

9. The ultrasound transducer of claim 1, wherein
the second color is different from a color having a medical meaning.

10. The ultrasound transducer of claim 1, wherein
the attachment portion has the concave shape configured to attach to the convex portion of the puncture adapter, or the attachment portion has the convex shape configured to attach to the concave portion of the puncture adapter.

11. An ultrasound diagnostic apparatus, comprising:
a body configured to transmit and receive ultrasound waves, the body having a first color;
an attachment portion having a concave shape or a convex shape, the attachment portion configured to attach to a convex portion or a concave portion provided on a puncture adapter; and
at least one guide mark provided on a surface of the body, the at least one guide mark having a second color different from the first color;
an image generating unit configured to generate an ultrasound image based on the ultrasound waves received by the body; and
a display configured to display the ultrasound image generated by the image generating unit.

12. The ultrasound diagnostic apparatus of claim 11, wherein
the at least one guide mark is colored on the concave shape or the convex shape of the attachment portion.

13. The ultrasound diagnostic apparatus of claim 11, further comprising:
an attachment surface provided on the body, the attachment surface configured to attach to the puncture adapter,
wherein the at least one guide mark is provided on the attachment surface, the at least one guide mark indicating a position of the attachment portion.

14. The ultrasound diagnostic apparatus of claim 13, wherein
the at least one guide mark has an arrow shape, the arrow shape of the at least one guide mark indicating a position of the attachment portion.

15. The ultrasound diagnostic apparatus of claim 11, further comprising:
an attachment surface provided on the body, the attachment surface configured to attach to the puncture adapter,
wherein the at least one guide mark is provided on the attachment surface, the at least one guide mark indicating a position of the attachment surface.

16. The ultrasound diagnostic apparatus of claim 15, wherein
the at least one guide mark is colored on an edge of the attachment surface or an inside edge of the attachment surface.

17. The ultrasound diagnostic apparatus of claim 11, wherein
more than one attachment portion and more than one guide mark are provided on the body, each guide mark depicting a number, the number indicating an order of attachment of the attachment portion, or the number indicating an order of attachment of multiple parts of the puncture adapter.

18. The ultrasound diagnostic apparatus of claim 11, further comprising:
an attachment surface provided on the body, the attachment surface configured to attach to the puncture adapter,
wherein the body comprises an insertion unit configured to insert into a cavity, and a grip unit configured to be held by an operator; and
the at least one guide mark is provided on a surface of the insertion unit.

19. The ultrasound diagnostic apparatus of claim 11, wherein
the second color is different from a color having a medical meaning.

20. The ultrasound diagnostic apparatus of claim 11, wherein
the attachment portion has the concave shape configured to attach to the convex d configured to attach to the concave portion of the puncture adapter.
